(19) United States
(12) Patent Application Publication
(10) Pub. No.: US 2010/0215601 A1
(43) Pub. Date: Aug. 26, 2010

(54) Glow in the dark nail polish

(76) Inventor: Sandra L. Abrahamian, Enfield, CT (US)

Correspondence Address: Sandra L. Abrahamian, 164 Fox Hill Lane, Enfield, CT, 06082, US

(21) Appl. No.: 12/380,069

(22) Filed: Feb. 23, 2009

(51) Int. Cl. A61K 8/60

(52) U.S. Cl. 424/61

(57) ABSTRACT

Glow in the dark nail polish for individuals who desire to have something more than their everyday nail polish selection. Applying a polish that will glow in the dark is not only different then the polish offered today, but it also would allow the user to enjoy another side of their nail polish. The user can have fun, be noticed and enjoy quality polish that would be available in several different colors and shades including the primary colors, black and white that will glow in the dark.

### BACKGROUND OF THE INVENTION

1. Field of the Invention

The present invention relates to nail polish that glows in the dark.

2. Description of the Prior Art

The prior art has not recognized the desire individuals have in wanting more in a nail polish. The prior art addresses different colors and shapes of color. When an individual colors their nails, the nails will look beautiful during the day but go unnoticed at night. The prior art is ‘what you see is what you get’. With the glow in the dark nail polish, the nails not only get notice during the day, but also get noticed at night. The user should have the option to select a nail polish that offers more than just different colors. Individuals may enjoy the ability to purchase a nail polish that will not only offer great colors but also offer the ability to glow in the dark. The glow in the dark nail polish will solve the issue of not really seeing the beauty of the nails at night by making a nail polish that glows in the dark and allowing the nails to be seen in a whole new light or lack thereof.

The pertinent prior art will be discussed in the order of their perceived relevance to the present invention.

U.S. Pat. No. 5,370,866 issued on Dec. 6, 1994, to Christopher C. Frankfurt et al. describes a colorless or colored nail polish composition containing aramide fibers for improved adhesion and strength of the nail polish on the nails.

U.S. Pat. No. 2,995,459 issued on Aug. 8, 1961, to Saul Soloway describes a method of coloring nacreous (mother-of-pearl) materials and the fingernail lacquer product compositions which can have gold and silver containing compounds.

U.S. Pat. No. 4,301,046 issued on Nov. 17, 1981, to Mitchell L. Schlossman describes a universal nail polish composition containing polyester resin.

U.S. Pat. No. 4,740,370 issued on Apr. 26, 1988 to Joseph R. Faryniarz et al. describes a process for preparing nail coating compositions containing pigment greater than 2% by weight and a Newtonian viscosity not greater than about 600 centipoises. The mill base and pigments are milled in high-energy milling equipment.

U.S. Pat. No. 1,974,466 issued on Sep. 25, 1934, to Preston W. Marshburn describes a nail polish package having a detachable chart of nail polish colors in graduated tints.

U.S. Pat. No. 5,778,901 issued on Jul. 14, 1998, to Sandra L. Abrahamian describes a personal color mixing nail polish kit.

The prior art does not suggest that a glow in the dark nail polish would be desired among those who wear nail polish and want their nails to be noticed at night as well as during the day.

None of the above inventions and patents, taken either singly or in combination is seen to describe the invention as claimed.

### SUMMARY OF THE INVENTION

Accordingly, it is a principal objective of the invention to provide glow in the dark nail polish.

It is another objective of the invention to provide different colors and shades of glow in the dark nail polish including the primary colors.

It is a further object of the invention to provide improved elements thereof in the glow in the dark nail polish described which is dependable and fully effective in accomplishing its intended purpose.

These and other objects of the present invention will become readily apparent upon further review of the following specification and drawings.

### BRIEF DESCRIPTION OF THE DRAWINGS

FIG. 1 is a perspective view of one nail polish bottle, containing glow in the dark nail polish, a perspective view of one nail polish brush, a perspective view of glow in the dark nail polish being applying to a fingernail and a perspective view of a finished fingernail that has the appearance of glowing in the dark.

Similar reference characters denote corresponding features consistently throughout the attached drawing.

### DETAILED DESCRIPTION OF THE PREFERRED EMBODIMENT

The present invention provides nail polish that glows in the dark. Today, individuals want choices and desire different ways to enjoy what they do everyday that go beyond the usual. Therefore, an effort has been made in the present invention to respond to the desires of wanting more in a nail polish. The user can select a polish from several colors and shades including the primary colors, black and white, which simply has the ability to glow in the dark.

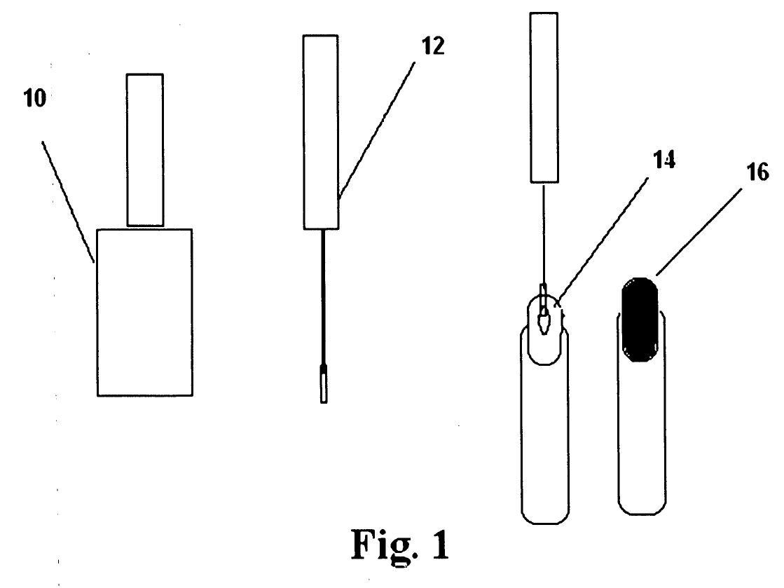

In FIG. 1, a nail polish bottle 10 of ‘glow in the dark’ nail polish is provided. A further view shows a nail polish brush 12, with the polish 14 being applied to a fingernail, and the finished nail polished 16 appearing to glow in the dark.

The nail polish may or may not be based on some of the following acetone, ethyl acetate, propyl acetate and/or butyl acetate with smaller amounts of nitrocellulose and isopropyl alcohol.

It is to be understood that the present invention is not limited to the sole embodiment described above, but encompasses any and all embodiments within the scope of the following claims.

I claim:

1. Nail polish that glows in the dark.

2. Nail polish that glows in the dark, according to claim 1, available in several different colors and shades including the primary colors, black and white.
